(19) United States
(12) Patent Application Publication
(10) Pub. No.: US 2013/0075309 A1
(43) Pub. Date: Mar. 28, 2013

(54) Self-Test of a Dual-Probe Chlorine Sensor for a Hemodialysis System

(71) Applicant: Nelson Environmental Technologies, Inc., McAllen, TX (US)

(72) Inventors: Burke A. West, McAllen, TX (US); Teng Beng Koay, McAllen, TX (US); Jeremy D. Brown, Rosharon, TX (US)

(73) Assignee: NELSON ENVIRONMENTAL TECHNOLOGIES, INC., McAllen, TX (US)

(21) Appl. No.: 13/626,653

(22) Filed: Sep. 25, 2012

Related U.S. Application Data
(60) Provisional application No. 61/539,155, filed on Sep. 26, 2011.

(51) Int. Cl. A61M 1/34

(52) U.S. Cl. 210/85; 210/96.2

(57) ABSTRACT

In a hemodialysis system, a microprocessor periodically controls the injection of a quantity of a halogen solution, preferably an iodine solution, to trigger an event alarm. At a predetermined time, the solution is circulated into a chlorine-specific sensor probe to the point of exceeding a threshold on the sensor system. This event is then recorded and a chlorine alarm event light is illuminated. After the slug of solution is flushed from the system, the alarm clears but the event light remains lit. Then, the next time an operator arrives to operate the hemodialysis system, she can verify that the monitor recorded a chlorine event since the previous day. She then resets the event light.

This application claims the benefit of U.S. Provisional Patent Application No. 61/539,155 filed Sep. 26, 2011.

### BACKGROUND OF THE INVENTION

The present invention relates generally to the field of hemodialysis apparatus, and more particularly to a system for monitoring the presence of chlorine in the water supply subsystem and, more particularly, to a dual-probe, chlorine specific detector that periodically verifies the proper functioning of the detector with a self-test function by introducing a “slug” of a iodine solution to the detector.

### FIELD OF THE INVENTION

As described in U.S. Pat. No. 4,828,693 to Lindsay et al., hemodialysis machines are used to extract waste from human blood of patients having kidney failures or disorders. Such machines use a dialyzer in which the blood flows through one chamber and a dialysate solution flows through another chamber separated from the first chamber by a membrane. The dialysate picks up metabolic waste products and ultrafiltrate from the blood passing through the dialyzer. In many such machines, incoming water is first treated externally to the machine to remove impurities, and thereafter pressure controlled, filtered, heated, and applied to a proportioning pump which is also connected to a supply of concentrated dialysis solution. The proportioning pump produces a carefully controlled dialysate solution from the water and concentrate. For example, a 34 parts water and 1 part dialysate concentrate is typical. The dialysate may be applied to a flow controller or other means which controls the rate of flow of the dialysate through the dialyzer.

One concern in hemodialysis today is the possible exposure of patients to chlorine. The chlorine can accidentally remain in or be introduced into the patient water by at least two ways. First, the chlorine can be introduced into the storage tank through the reverse osmosis unit (RO) product water stream. This is possible if chlorine is in the feed water to the RO. If the chlorine is in the form of free chlorine, the RO membranes will remove between 40% and 60% of the chlorine. That means that 40% to 60% of the chlorine will pass the RO and go directly to the water storage tank. If the chlorine is in the form of combined chlorine (or chlorinated solids), the chlorine can pass through any leaks in or around the membranes. Although rare, it is for this reason that RO machines are not considered effective disinfection devices.

Second, the chlorine can remain after the patient water loop has been disinfected. This will occur if the loop is not adequately rinsed after disinfection. This sometimes happens, even with the best of training of operators.

Chlorine in the municipal water supply today in the United States can come in several forms: free chlorine, monochloramines, naturally-occurring multichloramines, other dissolved combined chlorines, and chlorinated organics and other solids. Free chlorine is the easiest to remove. It reacts with carbon on contact. The rest of the dissolved chlorines are removed by adsorption in the carbon bed. Because the adsorption process is time dependent, it is important that the influent water have sufficient contact time with the carbon.

The most problematic chlorines to remove are in the form of chlorinated solids. These solids can be partially removed in the carbon bed by physical filtration (sedimentation). The solids get captured in the porous carbon media and are backwashed out with the rest of the sediments collected by the carbon filters. Unfortunately, during the back-washing of the filter beds these chlorinated solids enter the bottom of the bed. The carbon bed is expanded by 50% allowing for easy entry of the chlorinated solids to enter the complete bed. Then after a 4-minute (typical) settling period, the carbon bed is forward-rinsed, compacting the bed and trapping the chlorinated solids in it. Many will gradually rinse out causing false-positives when checking for chlorine at the start of the work day.

Because of the risk of having any of these chlorine forms in the feed water reach the RO machine, they need to be effectively removed before reaching the RO. The current practice of checking for chlorine after the first chlorine (worker) filter using DPD (N,N diethyl-p-phenylene diamine) is good as it will react with all forms of chlorine. If chlorine is detected, testing with test strips will determine if the chlorine is dissolved (detected) or solids (not detected).

Because of the criticality of the chlorine monitoring portion of the hemodialysis system, it is equally critical to ensure that the monitoring portion of the system is indeed functioning. The present invention is directed to fulfilling this long felt need in the art.

### SUMMARY OF THE INVENTION

The present invention addresses these and other needs in the art of hemodialysis by providing a microprocessor controlled self-test subsystem. At a predetermined time, a quantity, referred to herein as a “slug” of a solution including a halogen, preferably iodine but alternatively chlorine, is circulated into the chlorine sensor probe to the point of exceeding a threshold on the sensor system. This event is then recorded and a chlorine alarm event light is illuminated. After the slug of solution is flushed from the system, the alarm clears but the event light remains lit. Then, the next time an operator arrives to operate the hemodialysis system, she can verify that the monitor recorded a chlorine event since the previous day. She then resets the event light.

These and other features and advantages of this invention will be readily apparent to those skilled in the art.

### BRIEF DESCRIPTION OF THE DRAWINGS

So that the manner in which the above recited features, advantages and objects of the present invention are attained and can be understood in detail, more particular description of the invention, briefly summarized above, may be had by reference to embodiments thereof which are illustrated in the appended drawings.

FIG. 1 is a schematic diagram of a hemodialysis water treatment system wherein the present invention filed application.

FIG. 2A is a top view of a dual-probe chlorine monitor in the form of a pre-treat cell block in accordance with this invention.

FIG. 2B is a top view of a dual-probe chlorine monitor in the form of a post-treat cell block in accordance with this invention.

### DETAILED DESCRIPTION OF A PREFERRED EMBODIMENT

The present invention protects the safety of hemodialysis patients from accidentally being subjected to chlorine. Hemodialysis clinics go to great lengths through rigid protocols to ensure that the likelihood of this happening is very small. However, without a chlorine monitor in place in the patient water delivery loop, there is no way to know for sure that chlorine is not present in the water other than at the beginning of the shift and this relies upon the nurse performing the test correctly.

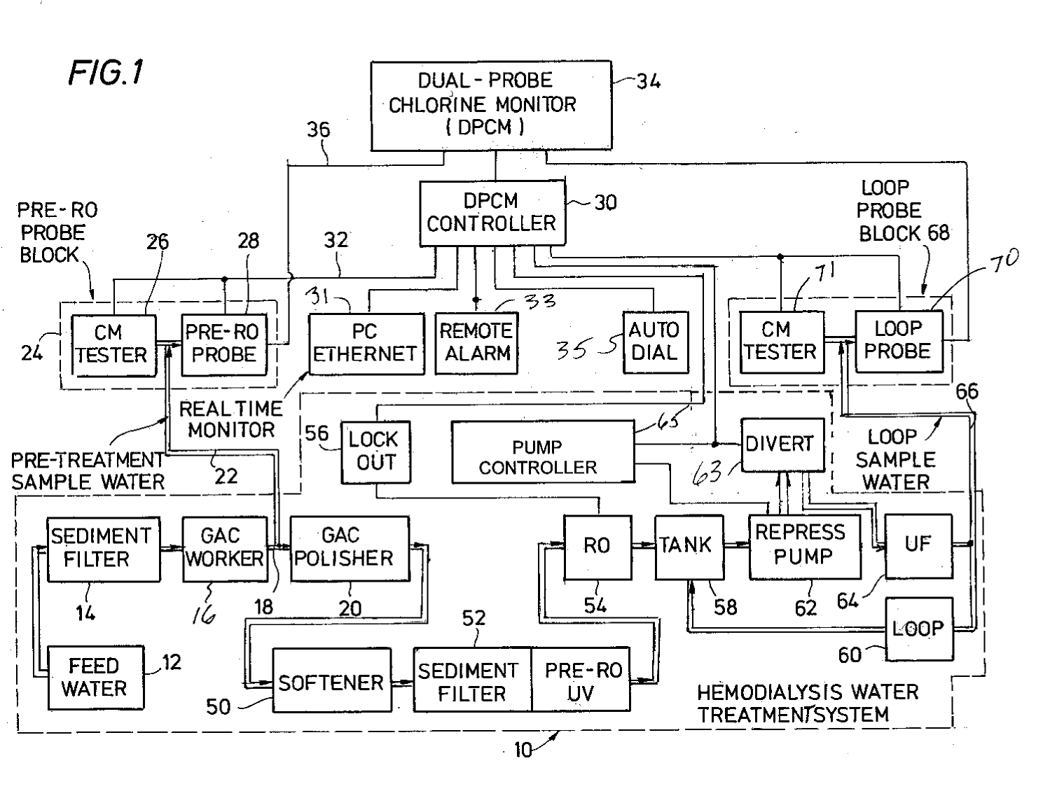

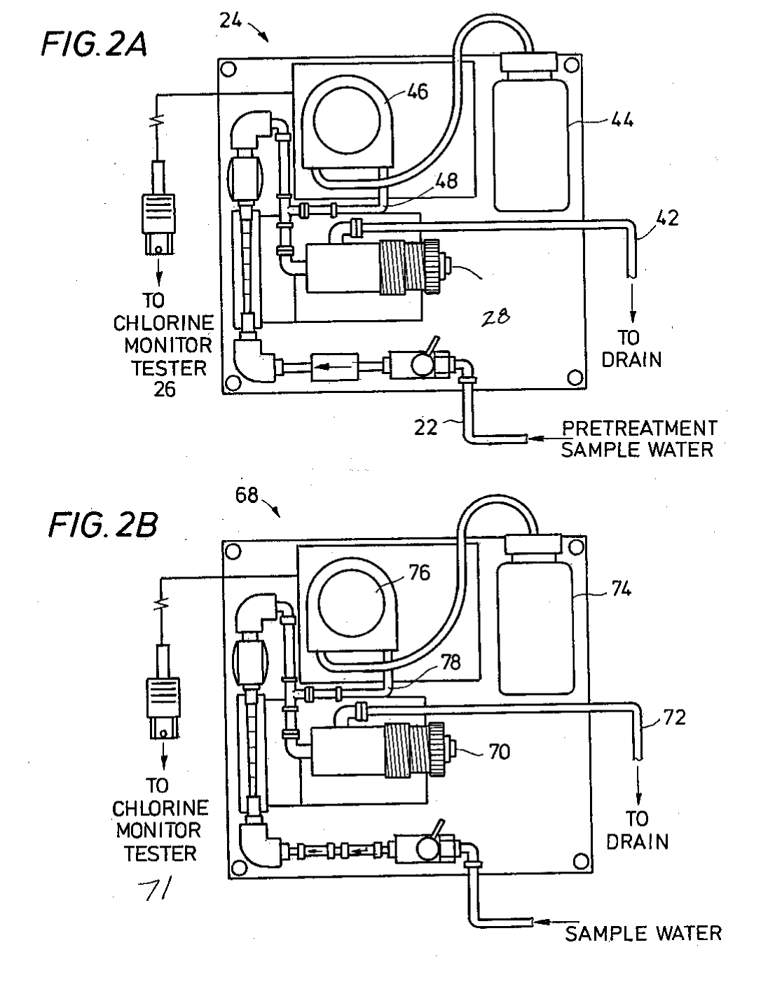

The present invention provides a dual-probe chlorine-specific monitor unit. FIG. 1 illustrates an overall block diagram of a hemodialysis water treatment system modified to accommodate the dual-probe of this invention. FIG. 2 shows how the monitor unit of the present invention interfaces with a hemodialysis water treatment system. The monitor unit includes a first probe positioned to detect combined chlorine in the patient water loop and alarms advising staff immediately of the problem, but it is also sensitive to other elements, including iodine. When connected to a divert system (or re-pressurizing pump controller), the contaminated water will be prevented from reaching the patient.

A second probe is placed in the post-treatment section after the worker carbon filter. If chlorine is detected, alarms activate and the pre-treatment lockout switch will de-activate the RO.

When monitoring for chlorine, it is critical to use chlorine-specific probes. The problem with using non-chlorine-specific monitors (secondary monitors) such as ORP is that other dissolved compounds in the water cause interference. This means that the chlorine in the water may be camouflaged (not be detected when it is actually present). ORP is pH and conductivity sensitive. The pH should be adjusted to within the range of 6.5 to 7.5 before initiating the ORP control. If the patient station water is going to be monitored, another major issue with ORP and pH presents itself. As the water gets purer, pH becomes less meaningful and more difficult to control. Separate protocols would need to be used for deionized water. Thus, using secondary methods for free chlorine detection adds unnecessary complexity as well as dangers to the process.

In order to ensure the proper functionality of the equipment, the system has both an automatic self-test and a manual self-test procedure. The system is set up to automatically inject a (preferably) iodine solution at a specific time, preferably during the night. This will cause the probe to register the halogen in the waste stream and trigger the alarms and event light. The alarms will silence once the iodine in the waste-stream is gone but the event light will remain on until manually reset. The system can also be tested by manually pressing the test button. The system will respond the same as the automatic test except that the alarms will not be activated (blocked for a predetermined time). If a loop divert or pretreatment lockout is connected, they will not activate during the test period.

The dual-probe chlorine monitor system includes an optional auto-dialer to call pager and telephone numbers giving a text or pre-recorded message. A terminal bar facilitates connections to remote alarms. The auto-dialer also monitors electrical power to the water treatment system as well as one more accessory such as a flood control monitor. All four channels of the auto-dialer have separate recorded messages and telephone numbers to dial.

### The Hemodialysis Water Treatment System

FIG. 1 illustrates a hemodialysis water treatment system 10. Feed water is introduced into the system 10 at a feed water inlet 12 in the conventional manner. The feed water is first filtered by a sediment filter 14 and then introduced to a granulated activated charcoal (GAC) worker 16. The (GAC) bed filter is filled with activated carbon that reacts with free chlorine and absorbs other chlorine compounds such as monochloramines that are in the influent water stream from the municipality water system. As the chlorine in its various states passes through the bed filter, it is removed from the water stream. An outlet line 18 from the GAC worker 16 provides an inlet to a GAC polisher 20 and also supplies a pretreatment sample line 22. The granulated activated carbon bed filter 20 is used as a redundant filter to the GAC worker 16 in order to help protect the system if for some reason the GAC worker 16 filter fails. The filters are identical; the first filter is called the worker as it removes the chlorine and second filter is called the polisher to remove any chlorine that should pass through the first filter. The line 22 feeds pretreatment sample water to a pre-RO (reverse osmosis) probe block 24. The pre-RO probe block is shown in simplified schematic form in FIG. 1 and is shown in greater preferred structural detail in FIG. 2.

The pre-RO probe block contains a chlorine monitor (CM) tester 26 and a pre-RO (chlorine specific) probe 28. Both the CM tester 26 and the pre-RO probe 28 are controlled by a dual-probe chlorine monitor (DPCM) controller 30 by way of a control line 32. The pre-RO probe 28 detects the level of chlorine in the pretreatment sample water and provides data to a DPCM monitor 34 by way of a data line 36. The controller 30 is also coupled to a real time monitor 31, a remote alarm 33, and an auto dial 35 as described in greater detail below.

As used herein, the term “chlorine specific” refers to a probe or other sensor that is specifically designed to detect chlorine, as opposed to a sensor such as an oxidation-reduction-potential (ORP) sensor that is used for many applications that can also be used as an indication of chlorine. However, the chlorine specific probe referred to herein will react with other chemicals also. For example, iodine, another halogen, can be detected by the probe. Because iodine is stable over time and chlorine in water is not, iodine is preferably used for the “bump test” of the present invention and not chlorine in order to do the daily testing of the probes. The iodine is only used to do a “bump test” to ensure the functionality of the equipment. The probes are calibrated for actual chlorine concentrations. Once a sample solution is periodically injected into the system, a latching event indicator indicates that the sensitive portion of the system of working satisfactorily.

Turning now briefly to FIG. 2, pretreatment sample water is supplied to the pre-RO probe block 24 by way of the line 22. The presence of chlorine in the sample water is sensed by a pre-RO probe sensor 28 and then the water flows out a drain 42. At a predetermined time each day, a slug of a testing solution is pumped from a solution bottle 44 by a pump 46 into a feed line 48 to provide a quantity of (preferably) iodine to the pre-RO probe sensor 28. If the pre-RO safety apparatus is functioning properly, an alarm condition is sent from the pre-RO probe block 28 to the DPCM 34. If the safety apparatus malfunctions for any reason and no alarm condition is sent, then corrective measures must be taken before the hemodialysis system is returned to service to treat patients.

Returning to FIG. 1, feed water is treated by the GAC polisher and then further conditioned by a softener 50 and a combination filter and pre-RO UV unit 52 before passing into a reverse osmosis (RO) unit 54. The sediment filter removes carbon fines and other solids in the water. This is necessary to ensure the efficacy of the UV disinfection unit. In the UV disinfection unit, water passes by a bulb that emits light with a wave length of 245 nanometers. This damages the DNA of microorganisms such that they cannot reproduce. If a contamination condition is detected in the DPCM controller 30, a lockout block 56 shuts off water to RO unit 54 within the system 10. If the condition of the water is satisfactory, the RO unit 54 provides the treated water to a storage tank 58. At this point, the water is available for use in treating a patient in the process of dialysis, as represented in FIG. 1 by a loop 60.

The patient is provided with treated water from the tank 58 by way of a pump 62 through a divert 63 and thence to a filter 64. The pump 62 is controlled by a pump controller 65.

In accordance with the present invention, a sample of the water is taken downstream of the filter 64 in a sample line 66. Sampled water flows in the sample line 66 to a loop probe block 68, as shown in FIG. 2. The loop probe block 68 comprises a loop probe sensor 70 and a chlorine monitor (CM) tester 71.

Treated sample water is supplied to the loop probe block 68 and the presence of chlorine in the sample water is sensed by a loop probe sensor 70. The water then flows out a drain 72. At a predetermined time each day, a slug of a testing solution is pumped from a solution bottle 74 by a pump 76 into a feed line 78 to provide a quantity of (preferably) iodine to the sensor 70. If the safety apparatus is functioning properly, an alarm condition is sent from the loop probe block 68 to the DPCM 34. If the safety apparatus malfunctions for any reason and no alarm condition is sent, then corrective measures must be taken before the hemodialysis system is returned to service to treat patients.

The present invention provides a simple design with a minimum number of components to minimize installation, with fewer components to fail. The system also includes fiberglass, water-resistant enclosures and the test controller can be locked. The system is easily installed into an existing water treatment system. The system is highly accurate, within approximately plus or minus 0.02 ppm, with repeatability of about plus or minus 0.01 ppm. Chlorine specific probes (not secondary methods of indication) are provided, as described above, with 25 ft. cables. No high maintenance chemical reagents are used (uses polarographic membranes). Daily automatic self-testing ensures proper equipment functionality.

The system has two probe blocks—one for each channel probe. Each probe block includes an injection pump with test solution container coupled into the waste-stream which has two check valves to prevent any back-flow of the chlorine test solution into the main loop stream. A pump lamp indicates the pump is activated during a test and calibration, as shown in FIG. 1. The patient loop block has a normally open solenoid valve to shut off the feed when the loop is being disinfected.

### DPCM Controller Features

The DPCM Controller provides many new, useful, and innovative features to enhance the usability of the system and to enhance patient safety. For example, as previously described, the controller 30 provides a periodic automatic test feature. During the automatic test, pre-treat lockout 56 and post-treat divert 63 are disabled. They are automatically enabled once the chlorine level returns to below the set point.

Other features include a manual test, which requires pressing and holding a manual test button for a preset period of time to initiate the manual test to prevent accidental actuation or non-qualified operator actuation. During manual test, the pre-treat lockout and divert are activated for a preset time interval and then disabled until the system's reading is below the set point. During calibration, if the calibration switch is left on, the system will alarm after a preset interval and the pump 62 will automatically turn off after a preset interval. During disinfection bypass, the alarm will sound after a preset interval if the switch is still on. There is a preset delay on the pump shut down after the disinfection bypass switch is turned off or the disinfection time and duration expire.

In addition, a pre-treatment manual bypass is accomplished by pressing the reset button for a preset period of time. The pre-treatment manual bypass overrides the alarm and pre-treat lockout in the pre-treatment. When in manual bypass, the alarm light will go from flashing to a solid light. A preset delay after a manual test is provided before the manual bypass can be used. During pre-treatment manual bypass, the alarm will automatically reset after the preset window has expired or the chlorine level is back below the set point.

A post-treatment manual bypass is accomplished by pressing the reset button for a preset interval and turning the disinfection bypass switch on and off. The post-treatment manual bypass causes the alarm light to go from flashing to a solid light. During post-treatment manual bypass, the alarm automatically resets after the preset window has expired or the chlorine level is back below the set point. Preset backwash window disables the pre-treat lockout, auto dialer, and alarm during backwashing and regenerating of the pretreatment tanks.

A loop disinfection window disables the divert/pump shut off during a scheduled disinfection. A preset delay on the divert/pump shuts down after the loop disinfection time expires. A data logger logs at a preset interval during regular service and logs at an accelerated rate during a real chlorine breakthrough and at a preset interval during testing (auto and manual).

A device backup battery powers an alarm notification on the controller and at the nurse's station. The auto dialer will also be activated in this event.

The principles, preferred embodiment, and mode of operation of the present invention have been described in the foregoing specification. This invention is not to be construed as limited to the particular forms disclosed, since these are regarded as illustrative rather than restrictive. Moreover, variations and changes may be made by those skilled in the art without departing from the spirit of the invention.

I claim:

1. A self-testing chlorine monitoring element in a hemodialysis water treatment system comprising an injection system adapted to subject a chlorine-specific probe to a known concentration of a solution bearing a halogen on a user-defined periodic basis.

2. The element of claim 1, wherein the halogen is iodine.

3. The element of claim 2, further comprising a latching event indicator.

4. The element of claim 2, further comprising a latching event manual reset.

5. The element of claim 2, further comprising alarms if manual switches are inadvertently left on after a specified period of time.

6. The element of claim 2, further comprising a lockout if chlorine detected at any time other than during the automatic testing cycle.

7. The element of claim 2, further comprising two chlorine probes controlled in a single chlorine monitoring system.

8. A self-testing chlorine monitoring system comprising:
a first, pretreatment probe block comprising a first chlorine-specific probe, a first source of a halogen solution, and means to periodically introduce a halogen solution from the source to the first chlorine-specific probe; and
a second, post-treatment probe block comprising a second chlorine-specific probe, a second source of a halogen solution, and means to periodically introduce a halogen solution from the source to the second chlorine-specific probe.

9. The system of claim 8, wherein the first and second sources retain a quantity of iodine solution.

10. The system of claim 8, further comprising a dual-probe chlorine monitor (DPCM) and a dual-probe chlorine monitor controller in data communication with the pretreatment probe block and the post-treatment probe block.

11. The system of claim 10, further comprising a pre-treat lockout and divert and wherein the controller is programmed to perform an automatic test, during which the pre-treat lockout and divert are disabled.

12. The system of claim 11, wherein the pre-treat lockout and the divert are automatically enabled once a sensed chlorine level is below a pre-determined set point.

13. The system of claim 10, wherein the DPCM controller includes a manual test, wherein the manual test requires pressing and holding a manual test button for a preset period of time to initiate the manual test to prevent accidental actuation or non-qualified operator actuation.

14. The system of claim 13, wherein during manual test, a pre-treat lockout and divert are activated for a preset time interval and then disabled until a system chlorine reading is below a set point.

15. The system of claim 10, wherein the DPCM controller includes a calibration, during which, if a calibration switch left on, the system alarms after preset interval and the pump will automatically turn off after a preset interval.

16. The system of claim 10, wherein further comprising a disinfection bypass, during which an alarm sounds after a preset interval if the switch is still on, and further comprising a preset delay on the pump shut down after the disinfection bypass switch is turned off or the disinfection time and duration expire.

17. The system of claim 10, wherein the DPCM controller includes a pre-treatment manual bypass actuated by pressing a reset button for a preset period of time.

18. The system of claim 17, wherein the pre-treatment manual bypass overrides the alarm and pre-treat lockout in the pre-treatment, and wherein the alarm light goes from flashing to a solid light.

19. The system of claim 18, further comprising a preset delay after a manual test before the manual bypass can be used.

20. The system of claim 19, further comprising an alarm which is automatically reset after a preset window has expired or the chlorine level is back below a set point.

21. The system of claim 10, wherein the DPCM controller includes a post-treatment manual bypass by pressing a reset button for a preset interval and turning a disinfection bypass switch on and off.

22. The system of claim 21, wherein the post-treatment manual bypass causes an alarm light to go from flashing to a solid light.

23. The system of claim 21, wherein during post-treatment manual bypass, an alarm will automatically reset after the preset window has expired or the chlorine level is back below the set point.

24. The system of claim 10, further comprising a preset backwash window which disables a pre-treat lockout, an auto dialer, and an alarm during backwashing and regenerating of pretreatment tanks.

25. The system of claim 10, further comprising a loop disinfection window which disables the divert/pump shut off during a scheduled disinfection.

26. The system of claim 25, further comprising a preset delay on the divert/pump shut down after the loop disinfection time expires.

27. The system of claim 10, further comprising a data logger which logs at a preset interval during regular service and logs at an accelerated rate during a real chlorine breakthrough and at a preset interval during automatic and manual testing.

28. The system of claim 10, further comprising a device backup battery which powers an alarm notification on the controller and at the nurse's station and activates an auto dialer.
